(19) United States
(12) Patent Application Publication
(10) Pub. No.: US 2004/0175312 A1
(43) Pub. Date: Sep. 9, 2004

(54) Diffuser for deodorizing products to be applied to vehicle vent-holes

(76) Inventor: Jolande Emilie Van Der Linden-Douw, Gouda (NL)

Correspondence Address: NOTARO AND MICHALOS, 100 DUTCH HILL ROAD, SUITE 110, ORANGEBURG, NY, 10962-2100, US

(21) Appl. No.: 10/382,672

(22) Filed: Mar. 6, 2003

(51) Int. Cl. A61L 9/00; A62B 7/08

(52) U.S. Cl. 422/306; 422/1; 422/5; 422/123

(57) ABSTRACT

It is herein described a diffuser for deodorizing products of the type consisting of an element provided with means apt to allow its application in correspondence with a vehicle vent-hole, the said diffuser showing openings for the inlet and outlet of an air flow apt to reach a deodorizing product introduced inside an envelope consisting of a semipermeable membrane apt to allow the passage of the perfumed oil molecules when it is reached by an air flow.

Means are provided apt to control the said air flow which reaches the said deodorizing product, consisting of a bored disk placed in contact with a wall wherein there are openings for the air flow inlet, the said disk can be rotated between a first position wherein it closes the said air inlet openings to a second position wherein it opens the said openings, between the said base and the said disk for the closure of the air inlet openings, a plurality of ports being provided drawn in a diffuser wall and apt to allow the outlet, throughout the environment, of the air flow coming from the vent-holes.

This invention proposes a diffuser for deodorizing products to be applied to vent-holes, in particular of vehicles and the like.

The diffuser is planned to be used with deodorants contained into a sachet made of a membrane which is waterproof but permeable to perfumed oil and when is reached by a blast of air, looses the perfume molecules.

The diffuser shows a plurality of adjustable openings for the air access, which air is directed to lick the membrane containing the deodorant to diffuse all over the environment.

The diffuser is preferably provided with a LED fed by a tab battery contained into the diffuser and can be activated by the lever which controls the opening of the air inlet vent-holes.

Different systems to perfume the interior of vehicles or the like are well known.

For example deodorizing products are known consisting of a support in a porous material impregnated with perfumed oil which, once exposed to the air, releases the perfume in the environment.

Containers are furthermore known which are filled with deodorizing granules or paste and are closed by a cover which is partially opened to allow the perfume to spread in the environment.

These containers of different types are generally fixed to vehicle parts such as the dashboard, through a bi-adhesive ribbon, Velcro® or another similar system.

Containers are also known which are addressed to be fixed in correspondence with the vent-holes of a car and which consist of a box provided with a grille or a series of inlet ports and a series of outlet ports on the opposite wall.

These containers are generally filled with granule deodorant so that the ventilation air provides to collect the perfume and to spread the same inside the vehicle.

Into this sector can be inserted the present invention which proposes a diffuser for deodorizing products to be applied to the vehicle vent-holes, the diffuser being provided to be used together with a deodorant packaging contained into an envelope made of a semipermeable membrane, which allows the passage of the perfumed oil molecules which, when the membrane is reached by a blast of air, get out by spreading in the environment.

The diffuser according to the invention shows on its bottom wall a plurality of adjustable air inlet vent-holes and is provided with a LED powered by a battery placed into the diffuser, which LED lights up when the air inlet vent-holes are open.

A device results therefrom which is particularly practical and efficient, simple to be assembled and which allows an easy replacement of the deodorant.

This invention will be now described in detail, by way of a non-restrictive example, with reference to the enclosed figures wherein:

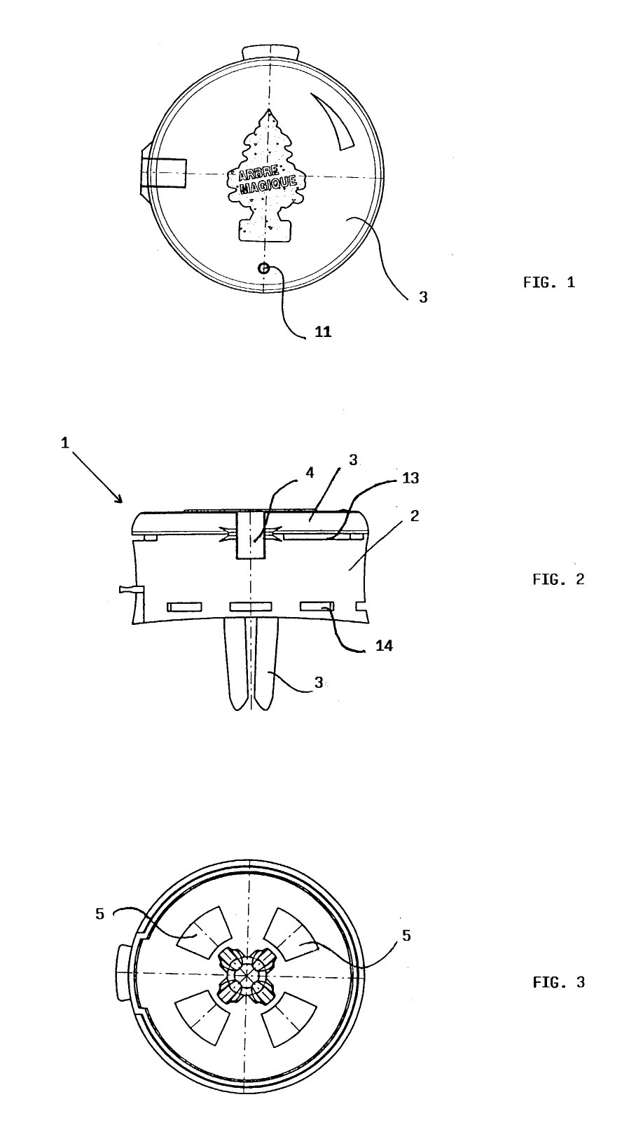

FIGS. 1, 2 and 3 are respectively a top view, a side view and a bottom view of the diffuser according to the invention with the cover closed;

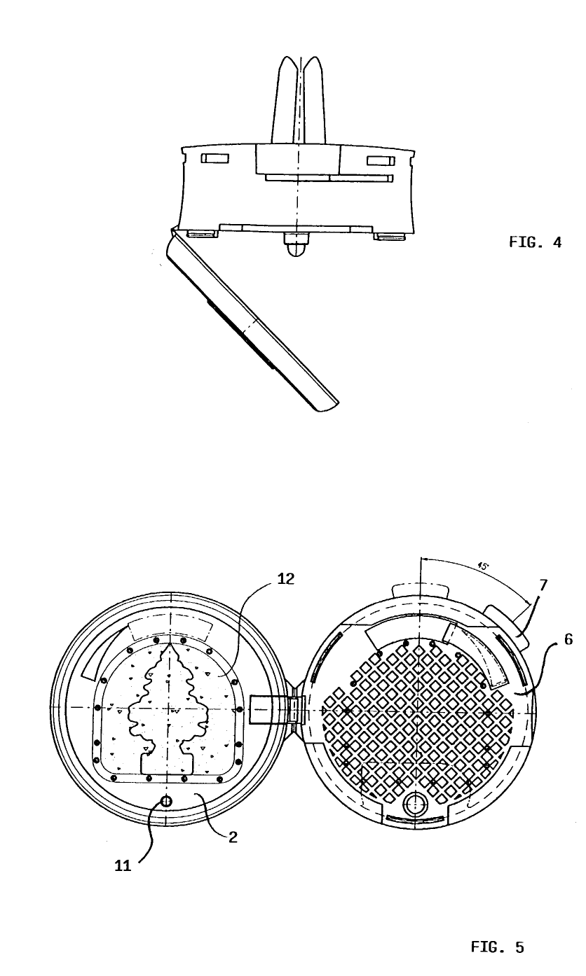

FIG. 4 is a side view of the diffuser with the cover while it is going to open;

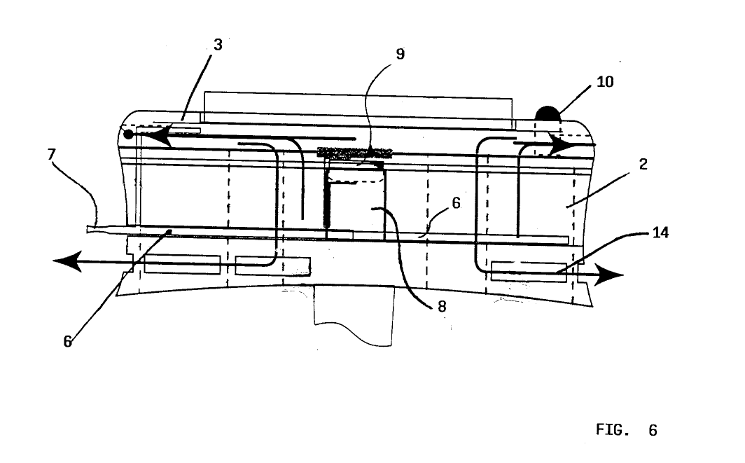

FIG. 5 is the top view of the diffuser with the cover opened and the envelope with the deodorant fit in;

FIG. 6 is the sectional view of the diffuser according to the invention with the air flow paths highlighted.

With reference to the figures, with reference 1 is indicated the diffuser, according to the invention, in its whole, which comprises a body 2, in this specific case having a round base, provided with coupling arms 15 of known type, for the fixing of the same on the vehicle vent-hole.

Upwardly the diffuser is closed by a cover 3 connected to the body 2 through a flexible plastic tab 4.

The body 2 shows at its base a series of openings 5 through which the air coming from the vehicle ventilation system can enter the diffuser.

A disk 6 or the like (FIG. 6) provided with openings similar to the openings 5 of the diffuser, rests on the bottom of the latter and shows a tooth or a projecting lever 7, through which it is possible to rotate the disk 6 inside the diffuser to carry the same from a position wherein the openings present in the disk match with the openings 5, allowing the free passage of the air, to a position wherein the disk closes the openings 5.

In the middle of the body 2 there is a cylindrical support 8 wherein a tab battery 9 is housed which feeds a LED diode 10 housed in the body 2 and visible through a port 11 present in the cover 3.

The feeding circuit of the diode 10 is preferably closed to feed the LED, when the disk 6 is rotated by acting on the lever 7 to carry the said disk in the opening position of the air inlet ports 5.

FIG. 5 shows the diffuser with the deodorant sachet, set out with reference 12, fitted inside.

On the lateral wall of the diffuser 2 (see FIG. 2) there are a series of air outlet openings, indicated with reference 13, placed in the upper part and an additional series of openings 14 placed near the base of the body 2, below the disk 6.

The operation takes place as follows:

The diffuser is put into a vent-hole with its base laid on the edge of the said vent-hole where it is held by the arms 15, which engage a support usually present inside the vent-holes.

The cover 3 is opened and the semipermeable envelope containing the deodorant is put in.

Once the cover is closed, the device is ready to be used.

With the disk 6 in a closing position of the openings 5, the air flow coming from the vent-hole slowly gets out laterally through the openings 14, at the base of the diffuser, without the perfume is spread in the environment.

By rotating the disk 6 through the lever 7, the openings 5 open and the air coming from the vehicle ventilation system licks the deodorant envelope, from which, through the semipermeable membrane, the perfume molecules get out and are spread in the environment always through the openings 13.

The activation of the perfume diffusion is also announced by the lighting of the LED 10, further to the closure of the feeding circuit consequent to the rotation of the disk 6.

Different forms of execution can be provided for with reference to the same idea of solution, but they have anyway to be considered all comprised within this invention.

1. A diffuser for deodorizing products of the type consisting of a body element apt to contain a certain quantity of deodorizing product and provided with means apt to allow its application in correspondence with a vehicle vent-hole, the said diffuser showing openings for the inlet and outlet of an air flow apt to reach this deodorizing product, characterized in that the said deodorizing product is introduced inside an envelope consisting of a semipermeable membrane apt to allow the passage of the perfumed oil molecules when it is reached by an air flow.

2. A diffuser for deodorizing products according to claim 1, characterized in that the air flow inlet openings are provided for in correspondence with the diffuser bottom wall and the perfumed air outlet openings in correspondence with the lateral wall, near the upper edge thereof.

3. A diffuser for deodorizing products according to claim 2, characterized in that means are provided apt to control the said air flow which touches the said deodorizing product.

4. A diffuser for deodorizing products according to claim 3, characterized in that the said means consist of a bored disk placed in contact with a wall wherein there are openings for the air flow inlet, the said disk can be rotated from a first position wherein it closes the said air inlet openings to a second position wherein it opens the said openings.

5. A diffuser for deodorizing products according to claim 3 or 4, charaterized in that between the said base and the said disk which closes the air inlet openings, there is a plurality of ports drawn in a diffuser wall, apt to allow the outlet, throughout the environment, of the air flow coming from the vent-holes.

6. A diffuser for deodorizing products according anyone of the previous claims, charaterized in that luminous means are provided to be activated when the diffuser is in operation.

7. A diffuser for deodorizing products according to claim 6, wherein the said luminous means consist of a LED visible through the diffuser cover and fed by a battery contained into the said diffuser, the feeding circuit of the said LED being closed when the said disk for the opening and closing of the air flow inlets is put in an opening position thereof.

8. A diffuser for deodorizing products according to anyone of the previous claims, characterized in that a cover is provided which can be opened to allow the replacement of the envelope of the deodorizing product.
